(19) United States
(12) Patent Application Publication
(10) Pub. No.: US 2025/0344938 A1
(43) Pub. Date: Nov. 13, 2025

(54) ENDOSCOPE

(71) Applicants: HANGZHOU XUNQI MEDICAL TECHNOLOGY CO., LTD., Hangzhou (CN); Hangzhou SKONSIN Health Technology Co., LTD., Hangzhou (CN)

(72) Inventor: Haitong LU, Hangzhou (CN)

(21) Appl. No.: 19/276,551

(22) Filed: Jul. 22, 2025

Related U.S. Application Data
(63) Continuation of application No. PCT/CN2024/119820, filed on Sep. 19, 2024.

(30) Foreign Application Priority Data
Apr. 2, 2024 (CN) 202410397140.0

(51) Int. Cl. A61B 1/00; A61B 1/04; A61B 90/00

(52) U.S. Cl. CPC A61B 1/00066; A61B 1/00096; A61B 1/00117; A61B 1/00119; A61B 1/00124; A61B 1/00128; A61B 1/00137; A61B 1/042; A61B 90/03; A61B 2090/034

(57) ABSTRACT

An endoscope includes a main body, a sealing protective member and a photoelectric assembly, where the main body includes a first shell and a tube body, a mounting cavity is within the first shell, and the tube body is connected to a front end of the first shell, and an interior of the tube body is communicated with the mounting cavity; the sealing protective member is arranged within the first shell and the tube body, an interior of the sealing protective member is through-going, and two ends of the sealing protective member that are through-going are hermetically connected to the mounting cavity and the tube body, respectively; and the photoelectric assembly includes a mounting box and an imaging unit connected to the mounting box, the imaging unit is arranged within the sealing protective member, and the mounting box is arranged within the mounting cavity.

### CROSS-REFERENCE TO RELATED APPLICATIONS

The present application is a continuation of International Application No. PCT/CN2024/119820, filed on Sep. 19, 2024, which claims priority to a Chinese Patent Application No. 202410397140.0, filed with China National Intellectual Property Administration on Apr. 2, 2024, entitled “Endoscope”. The aforementioned patent applications are hereby incorporated by reference in their entireties.

### TECHNICAL FIELD

The present application relates to the technical field of medical devices, and in particular, to an endoscope.

### BACKGROUND

Endoscopes are currently widely used medical devices for diagnosing and treating diseases. Endoscopes can be inserted into the human body through natural orifices of the human body or surgical incisions, providing a magnification effect on an observed area, and thus enabling precise localization and detailed observation of lesions.

Endoscopes must be inserted into the patient's body during use, coming into direct or indirect contact with the patient's body tissues during use. To enhance a hygiene of use, existing disposable endoscopes are discarded as a whole after use. Although this solves the problem of cross-infection, due to that the photoelectric assembly within endoscopes have a high cost and are easily contacted with the patient's body tissues during use, the photoelectric assembly is difficult to be reused, resulting in high diagnostic and treatment costs.

Therefore, there is an urgent need to address the technical problem of difficulty reuse of the photoelectric assembly within endoscopes in order to reduce costs.

### SUMMARY

The present application provides an endoscope to solve a technical problem that the photoelectric assembly in the endoscope is difficult to be reused, thus reducing production cost while ensuring the hygiene of use.

In order to achieve the above object, the present application provides an endoscope, including:
a main body, including a first shell and a tube body, where a mounting cavity is arranged within the first shell, and the tube body is connected to a front end of the first shell, and an interior of the tube body is communicated with the mounting cavity;
a sealing protective member, arranged within the first shell and the tube body, where an interior of the sealing protective member is through-going, and two ends of the sealing protective member that are through-going are hermetically connected to the mounting cavity and the tube body, respectively; and
a photoelectric assembly, including a mounting box and an imaging unit connected to the mounting box, where the imaging unit is arranged within the sealing protective member, and the mounting box is arranged within the mounting cavity.

In the endoscope provided by the present application, due to that the sealing protective member is arranged within the first shell and the tube body, two ends of the sealing protective member that are through are hermetically connected to the mounting cavity and the tube body, respectively, the imaging unit is arranged within the sealing protective member, and the mounting box is arranged within the mounting cavity, an effective isolation of the photoelectric assembly is achieved, thereby preventing contact between the photoelectric assembly and the patient's body tissue during surgery, and ensuring the photoelectric assembly not to be contaminated by a diseased body tissue, and thus guaranteeing the hygiene required for the reuse of the photoelectric assembly, and reducing the production cost of the endoscope.

In one possible implementation, the sealing protective member is a circumferentially closed tubular member to form an imaging channel within the sealing protective member so as to allow the imaging unit to pass therein.

In one possible implementation, a first end of the sealing protective member is arranged within the tube body and hermetically connected to an inner wall surface of a front end of the tube body, and a second end of the sealing protective member is hermetically connected to the mounting cavity.

In one possible implementation, a closed light-transmitting part is arranged in the front end of the tube body, and the first end of the sealing protective member is abutted against the inner wall surface of the front end of the tube body.

In one possible implementation, a connecting cap is arranged in the front end of the tube body, the connecting cap is hermetically connected to the front end of the tube body, the connecting cap has a light-transmitting hole, the light-transmitting part includes a light-transmitting sheet, the light-transmitting sheet is provided to cover the light-transmitting hole, and an edge of the light-transmitting sheet is hermetically connected to the light-transmitting hole.

In one possible implementation, a second end of the sealing protective member extends into the mounting cavity and is hermetically connected to an inner wall of the mounting cavity.

In one possible implementation, the mounting box includes a housing and a connecting seat, and a sealing cavity is within the housing, the connecting seat is arranged within the sealing cavity, the imaging unit includes a camera and a signal transmission line, two ends of the signal transmission line is connected to the camera and the connecting seat, respectively, and the connecting seat is used for electrically connecting the signal transmission line with the first shell.

In one possible implementation, a side wall of the housing is provided with a mounting hole for the signal transmission line to pass through, and an outer wall of the connecting seat is hermetically connected to the mounting hole.

In one possible implementation, the connecting seat includes a columnar part and a plate part connected to one end of the columnar part, the columnar part extends into the mounting hole, the columnar part is provided with a through-hole for the signal transmission line to pass through, the signal transmission line is hermetically connected to an inner wall of the through-hole, and a seal is provided between the columnar part and an inner wall of the mounting hole.

In one possible implementation, the mounting box further includes an elastic member being arranged within the sealing cavity, and the elastic member is abutted between an inner wall of the housing facing towards the mounting hole and the connecting seat.

In one possible implementation, the first shell is an integrally molded member, and a first channel, a second channel, a liquid inlet channel and a liquid outlet channel are arranged within the first shell, both the first channel and the second channel are communicated with an interior of the tube body, and at least part of a structure of the sealing protective member is arranged within the first channel; and the liquid inlet channel is communicated with the first channel, and the liquid outlet channel is communicated with the second channel.

In one possible implementation, a communicating port is arranged in the front end of the first shell, the tube body is hermetically connected within the communicating port, and the first channel and the second channel are both communicated with the communicating port.

In one possible implementation, the liquid inlet channel is communicated with a side of the first channel, and the liquid outlet channel is communicated with a side of the second channel; and/or
an inner duct is arranged within the tube body, one end of the inner duct is hermetically connected to the inner wall surface of the front end of the tube body and is communicated with an exterior of the tube body from the front end of the tube body, and other end of the inner duct extends into the second channel and is communicated with the liquid outlet channel.

In one possible implementation, the sealing protective member includes a first section, a second section and a third section connected in sequence, a part of the first section is exposed to an exterior of the first shell, the second section is within the first channel, the third section is within the tube body, and one end of the first channel away from the tube body is hermetically connected to an outer wall of the second section.

In one possible implementation, one end of the first shell away from the tube body forms a fixing seat, the mounting cavity is located within the fixing seat, a disassembly port is arranged at one end of the fixing seat opposite to the sealing protective member, and the main body further includes an end cap, and the end cap is capped over the disassembly port.

In one possible implementation, the end cap is hinged to an outer wall surface of the fixing seat, and a sealing ring is provided between the end cap and an outer peripheral surface of the fixing seat.

In one possible implementation, the end cap is connected to the fixing seat by a resilient part, and the end cap, the fixing seat, and the resilient part are integrally formed as a monolithic structure.

In one possible implementation, one end of the second channel away from the tube body extends into the fixing seat, and the main body further includes a waterproof sleeve, and the waterproof sleeve abuts between one end of the first channel away from the tube body and the end cap.

In one possible implementation, the main body further includes a second shell, the second shell includes a protective part, a protective cavity is within the protective part, at least a part of the first shell is arranged within the protective cavity, and the liquid inlet channel and the liquid outlet channel both pass through an inner wall of the second shell and extends to an exterior of the second shell.

The endoscope provided by the present application, by detachable setting of the photoelectric assembly, may prevent the photoelectric assembly from coming into contact with the patient's body tissue, ensuring the independence of the photoelectric assembly, and thus preventing the photoelectric assembly from being contaminated, so that the photoelectric assembly may be disassembled for secondary use, and conforms to medical standards, thereby helping to reduce production cost and ensuring safety and hygiene.

In the endoscope provided by the present application, the tube body is connected to the front end of the first shell, a liquid entered through the liquid inlet channel may smoothly enter a flow channel from the front end of the first channel, then flow along the flow channel, and enter an uterine cavity through a liquid outlet port; and when the liquid is discharged, it enters an interior of the inner duct through a liquid return port and is discharged through the liquid outlet channel, thereby achieving the flow of liquid.

In the endoscope provided by the present application, the inner duct is not only used for a surgical instrument to pass through for a surgical operation but also for a recovered liquid to pass through, resulting in a larger inner diameter of the inner duct, which facilitates the operation of the surgical instrument and also increases the speed of liquid recovery.

In addition to the technical problem solved by embodiments of the present application, the technical features constituting the technical solutions, and the beneficial effects brought by the technical features of the technical solutions described above, other technical problems that may be solved by a wearable endoscope provided by the embodiments of the present application, the other technical features included in the technical solutions, and the other beneficial effects brought by these technical features will be further detailed in the specific embodiments.

### BRIEF DESCRIPTION OF DRAWINGS

In order to more clearly illustrate technical solutions in the embodiments of the present application or in prior art, the following will be a brief introduction to accompanying drawings that need to be used in the description of the embodiments or prior art, and it will be obvious that the accompanying drawings in following description are some of the embodiments of the present application, and for those skilled in the field, other accompanying drawings may be obtained from these accompanying drawings without creative labor.

FIG. 1 is an exploded view of an endoscope provided by an embodiment of the present application.

FIG. 2 is another exploded view of an endoscope provided by an embodiment of the present application.

FIG. 3 is a three-dimensional structural diagram of a first shell of an endoscope provided by an embodiment of the present application.

FIG. 4 is a partial structural exploded view of an endoscope provided by an embodiment of the present application.

FIG. 5 is another exploded view of an endoscope provided by an embodiment of the present application.

FIG. 6 is a sectional view of an end cap in a closed state of an endoscope provided by an embodiment of the present application.

FIG. 7 is a sectional view of an endoscope provided by an embodiment of the present application.

FIG. 8 is an enlarged view of a structure at A in FIG. 7.

FIG. 9 is an enlarged view of a structure at B in FIG. 7.

FIG. 10 is a sectional view of an end cap in an open state of an endoscope provided by an embodiment of the present application.

FIG. 11 is a partial structural schematic diagram of a connecting cap and a tube body of an endoscope provided by an embodiment of the present application.

FIG. 12 is another partial structural schematic diagram of a connecting cap and a tube body of an endoscope provided by an embodiment of the present application.

FIG. 13 is a top view of a connecting cap of an endoscope provided by an embodiment of the present application.

FIG. 14 is a structural schematic diagram of a connecting cap and a light-transmitting sheet of an endoscope provided by an embodiment of the present application.

FIG. 15 is another partial structural schematic diagram of a connecting cap and a tube body of an endoscope provided by an embodiment of the present application.

FIG. 16 is a three-dimensional structural diagram of an end cap in an open state of an endoscope provided by an embodiment of the present application.

FIG. 17 is another three-dimensional structural diagram of an end cap in the open state of an endoscope provided by an embodiment of the present application.

FIG. 18 is a three-dimensional structural diagram of an end cap in a closed state of an endoscope provided by an embodiment of the present application.

Reference signs:
10—main body; 11—first shell; 111—first channel; 112—second channel; 113—liquid inlet channel;
114—liquid outlet channel; 115—fixing seat; 116—mounting cavity; 117—first protrusion; 118—flow channel; 12—second shell; 121—protective part; 122—handle part; 123—protective cavity; 13—end cap; 131—second protrusion; 132—operation hole; 133—sealing ring; 14—tube body; 141—inner duct;
20—sealing protective member; 21—imaging channel;
30—photoelectric assembly; 31—mounting box; 311—housing; 312—connecting seat; 3121—columnar part; 3122—plate part; 3123—through-hole; 313—mounting hole; 314—seal; 315—elastic member; 316—sealing cavity;
40—imaging unit; 41—camera; 42—signal transmission line; 421—sheath;
50—connecting cap; 51—light-emitting part; 52—light-transmitting sheet; 53—liquid return port; 54—liquid outlet port; 55—light-transmitting hole;
60—waterproof sleeve; 61—sleeve part; 62—blocking seal;
70—light guiding member;
80—control board; 81—control switch.

### DESCRIPTION OF EMBODIMENTS

To make the objectives, technical solutions, and advantages of the present application clearer, the technical solutions of the present application will be described clearly and completely with reference to the accompanying drawings of the present application. Obviously, the embodiments described are merely some examples of the present application and not all embodiments. All other embodiments obtained by those skilled in the art without creative work based on the embodiments of the present application fall within the scope of protection of the present application.

Endoscopes are widely used for in vivo diagnosis and treatment of living organisms, capable of probing into depths of narrow cavities in curved tube and observing areas that cannot be directly observed by human eye.

With existing endoscopes, photoelectric assembly is prone to come into contact with diseased body part during surgery, and it is difficult to ensure that the photoelectric assembly can be thoroughly disinfected. This renders the photoelectric assembly unsuitable for reuse, resulting in high endoscope cost and high surgical fee.

The endoscope provided by the embodiments of the present application will be described below with reference to the accompanying drawings using a hysteroscope as an example.

Before performing a hysteroscopic surgery, it is necessary to inject a liquid into the uterus to expand uterus, so as to examine the interior of the uterine cavity and acquire a clear image, and a surgical instrument passes through the hysteroscope to perform surgical operation on the patient's uterine cavity. Therefore, it is necessary to implement injection and drainage operations of a liquid such as uterine expansion liquid, which is introduced into the uterine cavity before surgery and is drained after examination is completed.

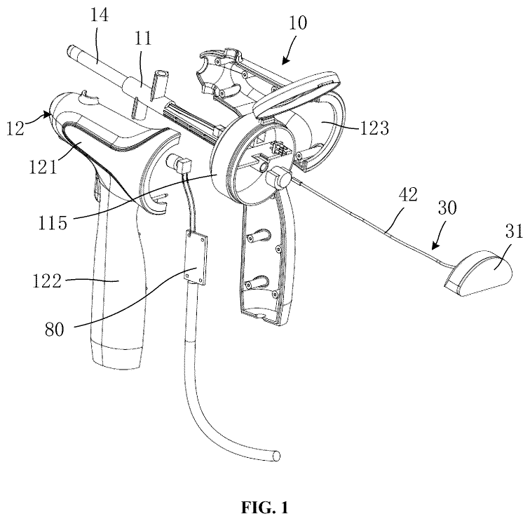

As shown in FIGS. 1, 2, and 3, the present application provides an endoscope including: a main body 10, a sealing protective member 20, and a photoelectric assembly 30, where the main body 10 includes a first shell 11 and a tube body 14, a mounting cavity 116 is within the first shell 11, and the tube body 14 is connected to a front end of the first shell 11, and an interior of the tube body 14 is communicated with the mounting cavity 116;

the sealing protective member 20 is arranged within the first shell 11 and the tube body 14, an interior of the sealing protective member 20 is through-going, and two ends of the sealing protective member 20 that are through-going are hermetically connected to the mounting cavity 116 and the tube body 14, respectively; and the photoelectric assembly 30 includes a mounting box 31 and an imaging unit 40 connected to the mounting box 31, the imaging unit 40 is arranged within the sealing protective member 20, and the mounting box 31 is arranged within the mounting cavity 116.

According to the endoscope provided by the present application, due to that the sealing protective member 20 is arranged within the first shell 11 and the tube body 14, two ends of the sealing protective member 20 that are through-going are hermetically connected to the mounting cavity 116 and the tube body 14, respectively, the imaging unit 40 is arranged within the sealing protective member 20, and the mounting box 31 is arranged within the mounting cavity 116, effective isolation of the photoelectric assembly 30 is achieved, thereby preventing contact between the photoelectric assembly 30 and the patient's body tissue during surgery, and ensuring the photoelectric assembly 30 not to be contaminated by diseased body tissue, and thus guaranteeing the hygiene required for the reuse of the photoelectric assembly 30, and reducing the production cost of the endoscope.

In the endoscope provided by the present application, as shown in FIG. 1, the first shell 11 may be injection molded, this helps to reduce assembly cost and lower overall cost.

The front end of the tube body 14 is inserted into the human or animal body during use to examine and treat the diseased site.

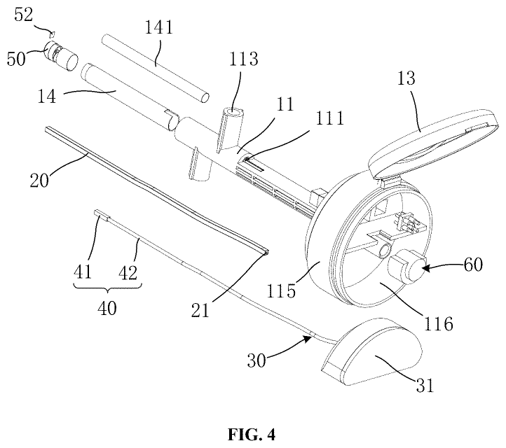

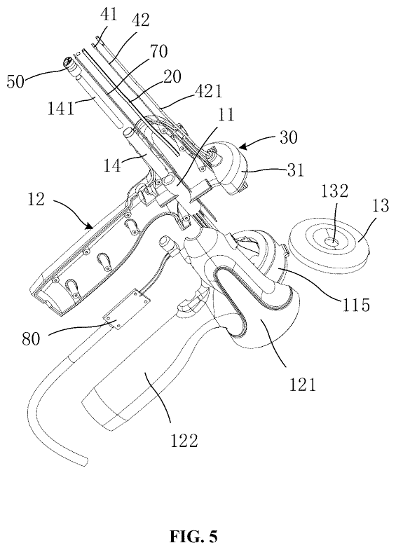

In one possible implementation, as shown in FIGS. 4 and 5, the sealing protective member 20 is a circumferentially closed tubular member to form an imaging channel 21 within the sealing protective member 20, allowing the imaging unit 40 to pass therein.

In one possible implementation, the sealing protective member 20 may be a square tube or a circular tube, which can be used for the imaging unit 40 to pass through. The sealing protective member 20 is used to protect the imaging unit 40, so that the imaging unit 40 is in a relatively sealed environment.

The sealing protective member 20 can be a slender tube and two ends of the sealing protective member 20 are through-going and the sealing protective member 20 are circumferentially closed, thereby preventing uterine expansion liquid from entering the imaging channel 21 inside the sealing protective member 20 through the circumference of the sealing protective member 20. Two ends of the sealing protective member 20 that are through-going being hermetically connected to the mounting cavity 116 and the tube body 14, respectively, may effectively prevent the uterine expansion liquid from entering the imaging channel 21 through the two ends of the sealing protective member 20, thereby protecting the imaging unit 40 located within the imaging channel 21 from contamination.

In one possible implementation, a first end of the sealing protective member 20 is arranged within the tube body 14 and hermetically connected to an inner wall surface of a front end of the tube body 14; and a second end of the sealing protective member 20 is hermetically connected to the mounting cavity 116.

The tube body 14 provides a protective function for the first end of the sealing protective member 20, effectively preventing the uterine expansion liquid from entering the sealing protective member 20 through the front end of the tube body 14, thereby protecting the imaging unit 40 located within the imaging channel 21 from contamination by the uterine expansion liquid or air within the uterine cavity. The second end of the sealing protective member 20 is hermetically connected to the mounting cavity 116, enhancing protection for the second end of the sealing protective member 20, which prevents bacteria from entering the imaging channel 21 through the second end of the sealing protective member 20 during surgery, thereby protecting the imaging unit 40 from contamination.

In one possible implementation, a closed light-transmitting part is arranged in the front end of the tube body 14, and the first end of the sealing protective member 20 is abutted against the inner wall surface of the front end of the tube body 14. The closed light-transmitting part may effectively prevent liquid or air from entering the interior of the sealing protective member 20 through the first end of the sealing protective member 20. This light-transmitting part allows light to pass through, enabling the imaging unit 40 to capture a clear image.

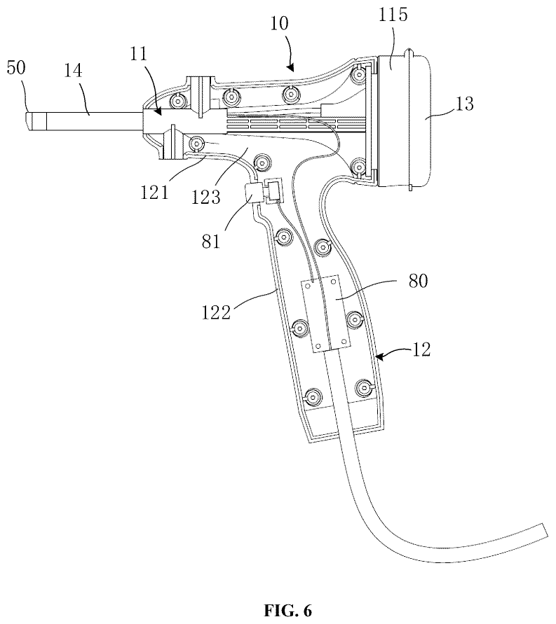

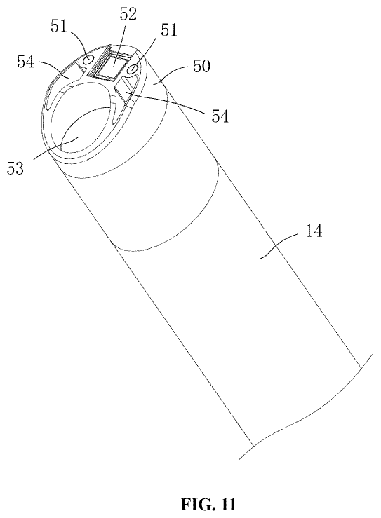

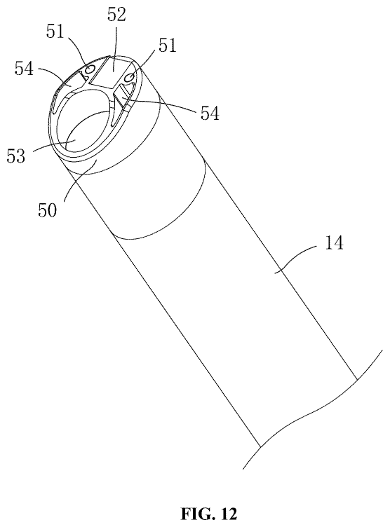

In one possible implementation, as shown in FIGS. 6, 7, and 9, a connecting cap 50 is arranged in the front end of the tube body 14, and the connecting cap 50 is hermetically connected to the front end of the tube body 14. As shown in FIGS. 11 and 12, the connecting cap 50 has a light-transmitting hole 55, the light-transmitting part includes a light-transmitting sheet 52, the light-transmitting sheet 52 is provided to cover the light-transmitting hole 55, and an edge of the light-transmitting sheet 52 is hermetically connected to the light-transmitting hole 55.

The connecting cap 50 is hermetically connected to the front end of the tube body 14, for example, by providing a tube sleeve between the connecting cap 50 and the front end of the tube body 14, with the two ends of the tube sleeve being respectively fitted over an outer peripheral surface of the connecting cap 50 and an outer peripheral surface of the front end of the tube body 14, and an adhesive being applied between the tube sleeve and the connecting cap 50, as well as between the tube sleeve and the front end of the tube body 14, to ensure a sealing effect.

As shown in FIGS. 12, 13, and 14, the hermetical connection between the first end of the sealing protective member 20 and the inner wall surface of the front end of the tube body 14 may be a sealing connection between the first end of the sealing protective member 20 and the inner wall surface of the connecting cap 50. For example, the hermetical connection between the first end of the sealing protective member 20 and the inner wall surface of the front end of the tube body 14 is achieved by applying an adhesive between the outer wall surface of the first end of the sealing protective member 20 and the inner wall surface of the connecting cap 50, or providing a sealing ring on the outer wall surface of the first end of the sealing protective member 20.

The light-transmitting hole 55 is a through hole, and the light-transmitting sheet 52 is provided to cover the light-transmitting hole 55 to block the light-transmitting hole 55 and prevent liquid or air from passing through the light-transmitting hole 55. The edge of the light-transmitting sheet 52 is hermetically connected to the light-transmitting hole 55, for example, by applying an adhesive at a position where the edge of the light-transmitting sheet 52 is connected with the light-transmitting hole 55, which not only enhances the mounting stability of the light-transmitting sheet 52 but also achieves a sealing effect.

The light-transmitting sheet 52 can be a transparent lens, such as a transparent resin sheet or silk-screened glass.

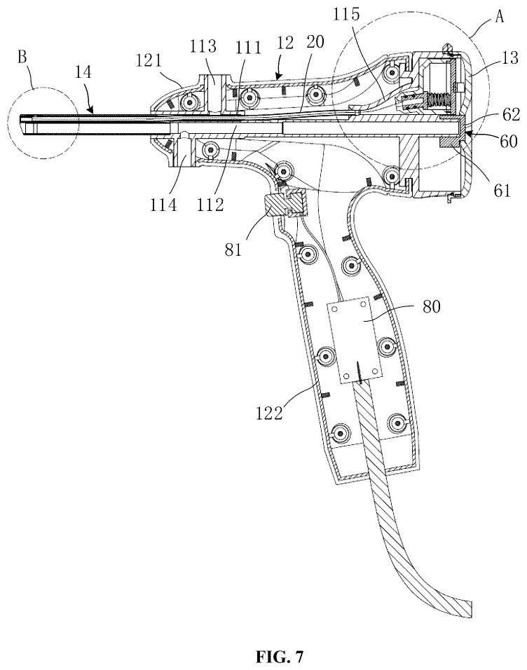

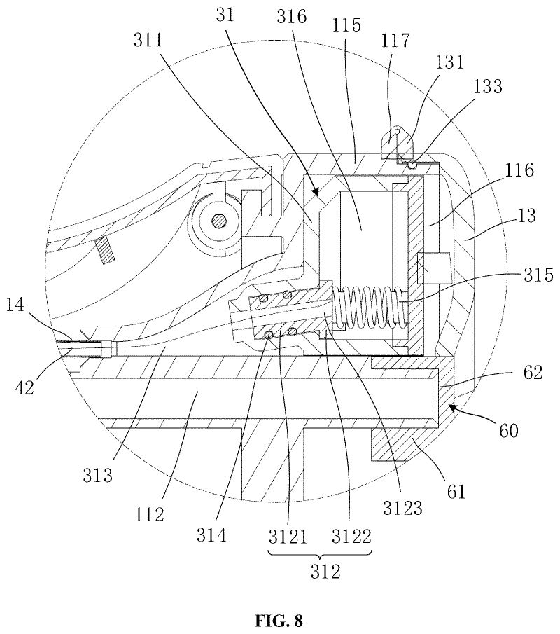

In one possible implementation, as shown in FIGS. 7 and 8, the second end of the sealing protective member 20 extends into the mounting cavity 116 and is hermetically connected to the inner wall of the mounting cavity 116.

For example, an adhesive may be applied between the outer wall of the second end of the sealing protective member 20 and the inner wall of the mounting cavity 116, thereby filling a gap between the outer wall of the second end of the sealing protective member 20 and the inner wall of the mounting cavity 116, preventing a liquid from passing through and achieving a sealing connection. Alternatively, a sealing ring may be provided between the outer wall of the second end of the sealing protective member 20 and the inner wall of the mounting cavity 116, and the sealing ring is compressed by the outer wall of the second end of the sealing protective member 20 and the inner wall of the mounting cavity 116, causing the sealing ring to deform, thereby filling the gap between the outer wall of the second end of the sealing protective member 20 and the inner wall of the mounting cavity 116, achieving a sealing effect. The sealing ring includes, but is not limited to, rubber, silicone or other material.

In one possible implementation, the mounting box 31 includes a housing 311 and a connecting seat 312, and a sealing cavity 316 is within the housing 311, the connecting seat 312 is arranged within the sealing cavity 316, as shown in FIGS. 4 and 8, the imaging unit 40 includes a camera 41 and a signal transmission line 42, two ends of the signal transmission line 42 are connected to the camera 41 and the connecting seat 312, respectively, the connecting seat 312 is used for electrically connecting the signal transmission line 42 with the first shell 11. The signal transmission line 42 is used to transmit a signal.

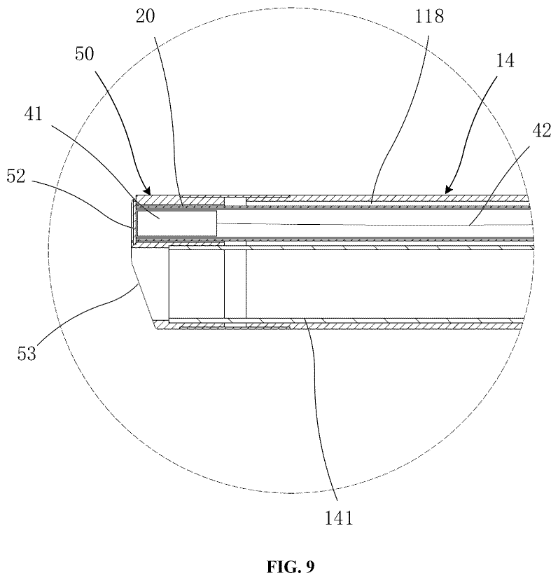

As shown in FIGS. 8 and 9, the signal transmission line 42 is externally covered with a sheath 421, which provides a protection effect and enhances the rigidity of the signal transmission line 42, ensuring the signal transmission line 42 to remain a stretched and straight state within the imaging channel 21.

A first terminal is arranged in an outer wall of the housing 311, and a second terminal is arranged in an inner wall surface of the mounting cavity 116. When the mounting box 31 is installed to the mounting cavity 116, the first terminal and the second terminal are positioned in correspondence and electrically connected. The first terminal and the second terminal can be electrically connected via a plug-in connection to ensure the stability of the electrical connection.

A third terminal is arranged in the connecting seat 312, and the third terminal is electrically connected to the first terminal, and the signal transmission line 42 is electrically connected to the third terminal of the connecting seat 312, thereby achieving an electrical connection between the signal transmission line 42 and the second terminal.

As shown in FIGS. 7 and 8, the housing 311 may further serve to protect the connecting seat 312.

In one possible implementation, as shown in FIGS. 7 and 8, a side wall of the housing 311 is provided with a mounting hole 313 for the signal transmission line 42 to pass through, and an outer wall of the connecting seat 312 is hermetically connected to the mounting hole 313. For example, an adhesive may be applied between the outer wall of the connecting seat 312 and the inner wall of the mounting hole 313, or a sealing ring may be provided between the outer wall of the connecting seat 312 and the inner wall of the mounting hole 313, in order to achieve a sealing effect.

In one possible implementation, the connecting seat 312 includes a columnar part 3121 and a plate part 3122 connected to one end of the columnar part 3121, the columnar part 3121 extends into the mounting hole 313, the columnar part 3121 is provided with a through-hole 3123 for the signal transmission line 42 to pass through, the signal transmission line 42 is hermetically connected to an inner wall of the through-hole 3123, and a seal 314 is provided between the columnar part 3121 and an inner wall of the mounting hole 313. The third terminal may be fixed to the plate part 3122.

The seal 314 may be a rubber ring. When compressed, the seal 314 undergoes elastic deformation, filling a gap between the columnar part 3121 and the inner wall of the mounting hole 313 to achieve a sealing effect.

The columnar part 3121 may be cylindrical in shape, and the mounting hole 313 may be a cylindrical hole.

The signal transmission line 42 being hermetically connected to the inner wall of the through-hole 3123 may be achieved, for example, by providing an adhesive between the signal transmission line 42 and the inner wall of the through-hole 3123, where the adhesive fills a gap between the signal transmission line 42 and the inner wall of the through-hole 3123 to achieve a sealing effect; or by providing a waterproof plug on an outer wall of the signal transmission line 42 and inserting the waterproof plug into the through-hole 3123, so as to fill the gap between the signal transmission line 42 and the inner wall of the through-hole 3123 by the elastic deformation of the waterproof plug, to achieve a stable sealing effect.

In one possible implementation, the mounting box 31 further includes an elastic member 315 arranged within the sealing cavity 316, and the elastic member 315 is abutted between an inner wall of the housing 311 facing towards the mounting hole 313 and the connecting seat 312.

In one possible implementation, the elastic member 315 may be a spring or an elastic block. Considering factors such as mounting error and manufacturing error, there may be an excessive gap or pressure between the first end of the sealing protective member 20 and the inner wall surface of the front end of the tube body 14, which may affect the use. The present application, by the elastic deformation of the elastic member 315, may keep the first end of the sealing protective member 20 in abutment against the inner wall surface of the front end of the tube body 14, and may prevent damage to the light-transmitting sheet 52 due to an excessive thrust of the first end of the sealing protective member 20 on the light-transmitting sheet 52, thereby enhancing safety of use and ensuring effective effectiveness of use.

Additionally, the elastic member 315, under the action of its own elastic thrust, always pushes the connecting seat 312 to move in a direction close to the mounting hole 313, thereby preventing the connecting seat 312 from moving away from the mounting hole 313, which is conducive to ensuring the sealing effect of the seal 314.

To enhance a stability of fixing the elastic member 315, a first convex post is arranged on the inner wall of the housing 311 facing the mounting hole 313, and a second convex post is arranged on a side of the plate part 3122 facing away from the columnar part 3121, and two ends of the elastic member 315 are sleeved on the first convex post and the second convex post, respectively, effectively preventing the elastic member 315 from falling off, thereby improving the mounting stability of the elastic member 315.

In one possible implementation, as shown in FIGS. 3 and 7, the first shell 11 is an integrally molded member, and a first channel 111, a second channel 112, a liquid inlet channel 113 and a liquid outlet channel 114 are arranged within the first shell 11, the first channel 111 and the second channel 112 are both communicated with an interior of the tube body 14, and at least a part of a structure of the sealing protective member 20 is arranged within the first channel 111; the liquid inlet channel 113 is communicated with the first channel 111, and the liquid outlet channel 114 is communicated with the second channel 112.

In one possible implementation, as shown in FIGS. 3 and 7, extend directions of the first channel 111 and the second channel 112 are in parallel, the liquid inlet channel 113 may be located on a side wall of the first channel 111, and the liquid outlet channel 114 may be located on a side wall of the second channel 112. The first channel 111 and the second channel 112 are separated inside the first shell 11, so that that the first channel 111 and the second channel 112 remain relatively independent.

In one possible implementation, a communicating port is arranged in the front end of the first shell 11, the tube body 14 is hermetically connected within the communicating port, and the first channel 111 and the second channel 112 are both communicated with the communicating port.

To prevent leakage at the connecting point between the front end of the first shell 11 and the tube body 14, an adhesive is applied between the communication port and the tube body 14. This not only enhances the connecting stability but also provides a sealing effect, thereby preventing leakage.

In one possible implementation, the liquid inlet channel 113 is communicated with a side of the first channel 111, and the liquid outlet channel 114 is communicated with a side of the second channel 112.

In one possible implementation, the liquid inlet channel 113 may be connected to an inlet valve, and the liquid outlet channel 114 may be connected to an outlet valve.

As shown in FIGS. 7 and 9, an inner duct 141 is arranged within the tube body 14, one end of the inner duct 141 is hermetically connected to the inner wall surface of the front end of the tube body 14 and is communicated with an exterior of the tube body 14 at the front end of the tube body 14, and other end of the inner duct 141 extends into the second channel 112 and is communicated with the liquid outlet channel 114.

The inner duct 141 may be connected to the inner wall surface at the front end of the tube body 14 via an adhesive, which not only enhances the fixing stability of the inner duct 141 but also creates a sealing effect between the front end of the tube body 14 and the inner duct 141.

A flow channel 118 formed between the inner duct 141 and the inner wall of the tube body 14. The flow channel 118 is communicated with the liquid inlet channel 113, and the liquid outlet channel 114 is communicated with the second channel 112.

A surgical instrument may be operated through the second channel 112 and the inner duct 141. A liquid may enter into the uterine cavity from the liquid inlet channel 113 by the flow channel 118. The liquid, when discharged, passes through the inner duct 141 into the liquid outlet channel 114 for discharge, thereby achieving dual channels for both instrument operation circulation and water circulation. The structure is simple and does not require complex assembly operations, which helps reduce the production cost of the endoscope.

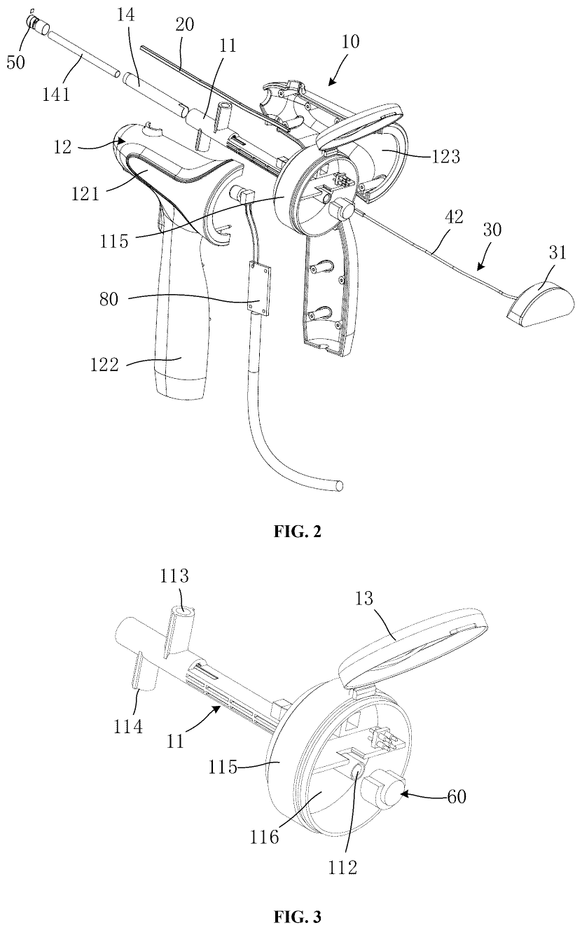

As shown in FIGS. 2, 11, and 12, the connecting cap 50 is provided with a liquid return port 53 and a liquid outlet port 54. The liquid return port 53 is communicated with an interior of the inner duct 141, and the liquid outlet port 54 is communicated with the flow channel 118. The liquid outlet port 54 is used to discharge the liquid from the flow channel 118 into the uterine cavity, while the liquid return port 53 is used to recover the liquid within the uterine cavity.

In one possible implementation, as shown in FIGS. 11 and 12, the liquid return port 53 may be circular or rectangular in shape.

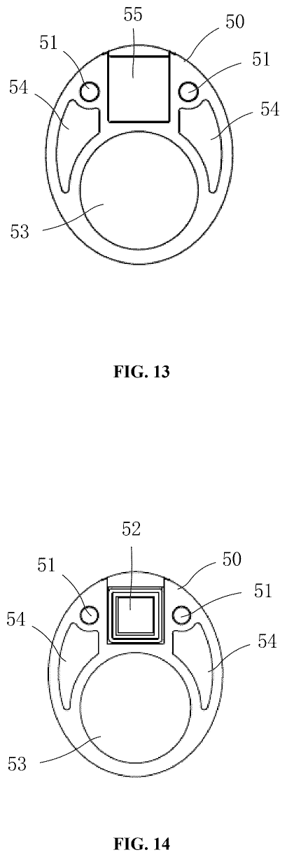

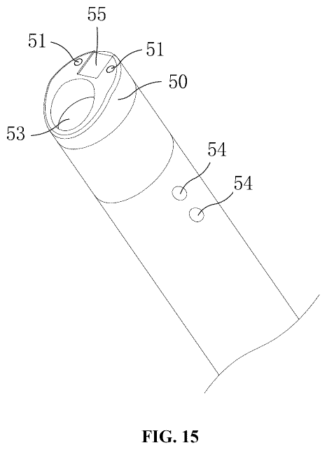

As shown in FIGS. 13 and 14, to improve liquid discharge efficiency, there may be two liquid outlet ports 54. The two liquid outlet ports 54 may be located on the front end surface of the tube body 14. Of course, as shown in FIG. 15, the liquid outlet port 54 may also be located on a side wall of the tube body 14, and the liquid outlet port 54 may be arc-shaped, circular, or other shapes, to enhance liquid discharge efficiency.

The connecting cap 50 further includes a light-emitting part 51. The endoscope further includes a light source, which may be a light-emitting diode (LED). A light guiding member 70 guides the light emitted by the light source to the light-emitting part 51, providing supplemental light to facilitate illumination of a part to be examined or treated. The light can pass through the light-transmitting sheet 52, to provide a favorable condition for the imaging unit 40 to capture an image and meet brightness requirements for imaging.

In one possible implementation, the light guiding member 70 can be an optical fiber, which has characteristics of low loss, light weight, and high fidelity, making it suitable for use in endoscopes.

In one possible implementation, as shown in FIGS. 7, 8, and 9, the sealing protective member 20 includes a first section, a second section and a third section connected in sequence, a part of the first section is exposed to an exterior of the first shell 11, the second section is arranged within the first channel 111, and the third section is arranged within the tube body 14, and one end of the first channel 111 away from the tube body 14 is hermetically connected to an outer wall of the second section. For example, an adhesive may be applied between one end of the first channel 111 away from the tube body 14 and the outer wall of the second section to achieve a sealing connection, which helps prevent leakage of the uterine expansion liquid from a position at one end of the first channel 111 away from the tube body 14.

In one possible implementation, the sealing protective member 20 may be integrally formed as a monolithic structure.

In one possible implementation, one end of the first shell 11 away from the tube body 14 forms a fixing seat 115, the mounting cavity 116 is located within the fixing seat 115, a disassembly port is arranged at one end of the fixing seat 115 opposite to the sealing protective member 20, and the main body 10 further includes an end cap, which is capped over the disassembly port.

In one possible implementation, the fixing seat 115 and the first shell 11 are integrally formed as a monolithic structure, eliminating the need for an assembly operation and helping to reduce assembly cost.

The mounting box 31 is pluggably provided within the mounting cavity 116, making mounting convenient and facilitating the removal of the photoelectric assembly 30.

In one possible implementation, as shown in FIGS. 4, 8, and 10, the end cap 13 is hinged to an outer wall surface of the fixing seat 115, and a sealing ring 133 is provided between the end cap 13 and an outer peripheral surface of the fixing seat 115.

The end cap 13 serves to protect the photoelectric assembly 30. When the end cap 13 is flipped upward to open the mounting cavity 116, the mounting box 31 of the photoelectric assembly 30 is exposed, facilitating the disassembly of the photoelectric assembly 30. As shown in FIG. 2, when the end cap 13 is flipped downward to close the mounting cavity 116, the photoelectric assembly 30 can be sealed within the mounting cavity 116.

The present application improves the sealing performance between the end cap 13 and the fixing seat 115 by providing a sealing ring 133 between the end cap 13 and the outer peripheral surface of the fixing seat 115.

In one possible implementation, as shown in FIGS. 4, 8, and 10, the end cap 13 and the fixing seat 115 may be connected with each other via a pin. For example, a first protrusion 117 is arranged in an outer wall of the fixing seat 115, and a second protrusion 131 is arranged in an outer side surface of the end cap 13. The pin is arranged in the first protrusion 117 and the second protrusion 131, enabling the end cap 13 to rotate relative to the fixing seat 115.

In one possible implementation, the end cap 13 is connected to the fixing seat 115 by a resilient part, and the end cap 13, the fixing seat 115, and the resilient part are integrally formed as a monolithic structure.

The resilient part may, for example, be a thin plate-like component with a certain degree of flexibility. The resilient part may, for example, be made of a polypropylene (PP) material, which can be repeatedly bent and is durable.

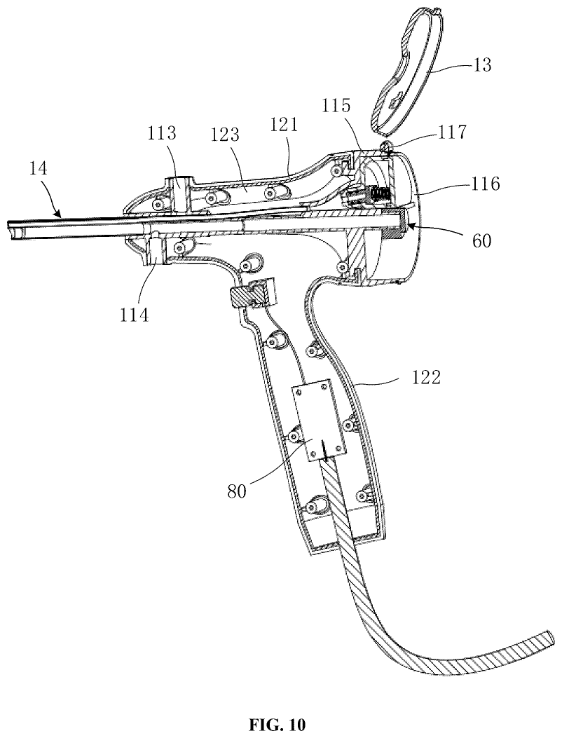

In one possible implementation, as shown in FIGS. 8 and 10, one end of the second channel 112 away from the tube body 14 extends into the fixing seat 115, and the main body 10 further includes a waterproof sleeve 60, which is abutted between one end of the first channel 111 away from the tube body 14 and the end cap 13.

The waterproof sleeve 60 includes a sleeve part 61 and a blocking seal 62, connected to the sleeve part 61. The sleeve part 61 is sleeved on the outer wall surface of the second channel 112, and the blocking seal 62 rests against one end of the first channel 111 away from the tube body 14. Since the liquid entering into the inner duct 141 through the liquid return port 53 will flow into the second channel 112, the provision of the waterproof sleeve 60 serves to seal the second channel 112.

The sleeve part 61 is arranged on the outer wall surface of the second channel 112 by a threaded connection between an inner wall of the sleeve part 61 and the outer wall surface of the second channel 112, or by an interference fit between the sleeve part 61 and the outer wall surface of the second channel 112.

In one possible implementation, the blocking seal 62 is a flexible member with a slit formed therein. By forming a slit in the blocking seal 62, a surgical instrument such as scalpel may enter the second channel 112 through the slit, while edges of the slit maintain contact with the surgical instrument, ensuring the sealing of the second channel 112.

In one possible implementation, the slit may, for example, be in the shape of a straight line.

In one possible implementation, the blocking seal 62 may, for example, be made of a rubber or silicone material with a certain degree of elasticity, and the blocking seal 62 may be a monolithic structure integrally molded with the sleeve part 61.

In one possible implementation, the waterproof sleeve 60 may also be fixed to a side of the end cap 13 facing the second channel 112. When the end cap 13 is closed, a compression force exerted by the end cap 13 on the waterproof sleeve 60 increases a pressing force between the waterproof sleeve 60 and the end of the second channel 112, thereby improving the sealing performance.

In one possible embodiment, as shown in FIGS. 1 and 2, the main body 10 further includes a second shell 12, the second shell 12 includes a protective part 121, a protective cavity 123 is within the protective part 121, at least a part of the first shell 11 is arranged within the protective cavity 123, and the liquid inlet channel 113 and the liquid outlet channel 114 both pass through an inner wall of the second shell 12 and extend to an exterior of the second shell 12.

The second shell 12 can serve to protect the first shell 11.

In one possible embodiment, as shown in FIGS. 5 and 6, the second shell 12 further includes a handle part 122, which is connected to the protective part 121. For example, the handle part 122 is connected to one side of the protective part 121, such that the second shell 12 generally resembles a pistol shape.

In one possible implementation, the handle part 122 and the protective part 121 may integrally formed as a monolithic structure.

In one possible implementation, as shown in FIGS. 1 and 2, the endoscope further includes a control board 80. The control board 80 may be arranged within the handle part 122. The control board 80 and the second terminal located on the inner wall surface of the mounting cavity 116 are electrically interconnected via a wire. The control board 80 is used to control an operation of the photoelectric assembly 30. The control board 80 may, for example, be a circuit board. As shown in FIGS. 6 and 7, the endoscope further includes a control switch 81, which may be arranged on the handle part 122. The control switch 81 is electrically connected to the control board 80.

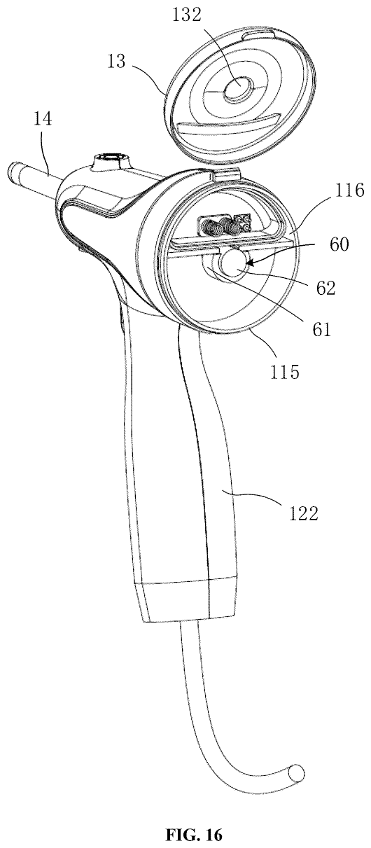

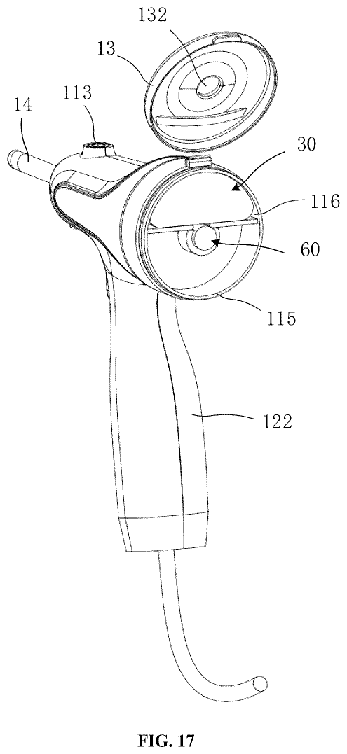

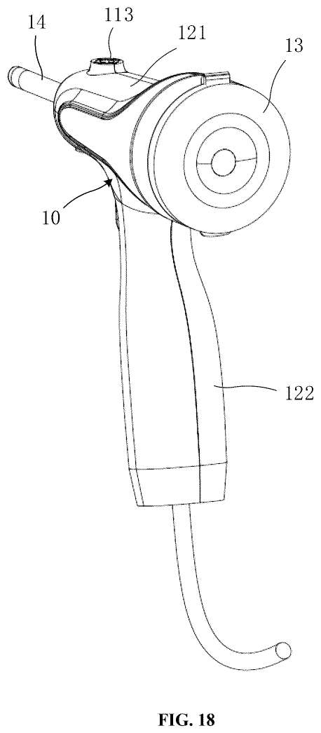

In one possible implementation, as shown in FIGS. 16, 17, and 18, the end cap 13 is provided with an operation hole 132, through which the blocking seal 62 is exposed. When the end cap 13 is closed, a surgical instrument such as scalpel may enter the second channel 112 through the slit in the blocking seal 62 to perform surgery, making it convenient to use.

The endoscope provided by the present application, considering the high cost of the photoelectric assembly 30, sets the photoelectric assembly 30 as a detachable one, avoiding the photoelectric assembly 30 contacting with the patient's body tissue and uterine expansion liquid, ensuring the photoelectric assembly 30 not to be contaminated. This allows the photoelectric assembly 30 to be disassembled for secondary use, addressing the issue of cross-infection, complying with medical standards, and helping to reduce production cost while ensuring safety and hygiene.

In the endoscope provided by the present application, the tube body 14 is connected to the front end of the first shell 11, the liquid entered through the liquid inlet channel 113 may smoothly enter the flow channel 118 from the front end of the first channel 111, then flow along the flow channel 118 and enter a uterine cavity through the liquid outlet port 54; and when the liquid is discharged, the liquid enters an interior of an inner duct 141 through a liquid return port 53 and is discharged through the liquid outlet channel 114. In this way, the flow of the liquid is achieved.

In the endoscope provided by the present application, the inner duct 141 is not only used for a surgical instrument to pass through to perform a surgical operation but also for a recovered liquid to pass through, resulting in a large inner diameter of the inner duct 141, which facilitates the operation of the surgical instrument and also increases the speed of liquid recovery.

In the description of the present application, it should be understood that the terms “center”, “length”, “width”, “thickness”, “top”, “bottom”, “up”, “down”, “left”, “right”, “front”, “back”, “vertical”, “horizontal”, “inside”, “outside”, “axial” and “circumferential” indicating the orientation or position relationships as used are based on the orientation or position relationships shown in the accompanying drawings, and are intended merely to facilitate the description of the present application and to simplify the description, and do not indicate or imply that the positions or elements referred to must have a specific orientation, or be constructed and operated in a particular manner. Therefore, they should not be construed as a restriction on the present application.

Furthermore, the terms “first” and “second” are only used for descriptive purposes and should not be understood as indicating or implying relative importance or implicitly indicating the quantity of the technical features indicated. Thus, the features defined by “first” and “second” can explicitly or implicitly include one or more such features. In the description of the present application, the meaning of “a plurality of” is at least two, such as two, three, etc., unless otherwise explicitly and specifically limited.

In the present application, unless otherwise explicitly defined and specified, terms such as “mount”, “connect”, “couple”, “fix”, etc. should be understood in a broad sense. For example, they can be a fixed connection, detachable connection, or integrated as a whole; can be a mechanical connection, an electrical connection or communicated with each other; can be a direct connection or indirect connection through an intermediate medium; or can be an internal connection between two components or an interaction relationship between two components. For those skilled in the art, the specific meanings of the above terms in the present application can be understood according to the specific circumstances.

In the present application, unless otherwise expressly defined and specified, the first feature being “above” or “below” the second feature may include that the first and second features are in direct contact, or the first and second features are not in direct contact, but are in contact through another feature between them. Moreover, the first feature being “above”, “on” and “over” the second feature includes that the first feature is directly above and diagonally above the second feature, or merely indicates that the horizontal height of the first feature is higher than that of the second feature. The first feature being “below”, “beneath” and “under” the second feature includes that the first feature is directly or diagonally below the second feature, or simply indicates that the horizontal height of the first feature is less than that of the second feature.

Finally, it should be noted that the above embodiments are only used to illustrate the technical solutions of the present application and not to limit the present application; although the present application is described in detail with reference to the foregoing embodiments, those skilled in the art should understand that it is still possible to modify the technical solutions recorded in the foregoing embodiments or to make equivalent substitutions for some or all of the technical features therein; and these modifications or substitutions do not take the essence of the corresponding technical solutions out of the scope of the technical solutions of the embodiments of the present application.

What is claimed is:

1. An endoscope, comprising:
a main body, comprising a first shell and a tube body, wherein a mounting cavity is within the first shell, and the tube body is connected to a front end of the first shell, and an interior of the tube body is communicated with the mounting cavity;
a sealing protective member, arranged within the first shell and the tube body, wherein an interior of the sealing protective member is through-going, and two ends of the sealing protective member that are through-going are hermetically connected to the mounting cavity and the tube body, respectively; and
a photoelectric assembly, comprising a mounting box and an imaging unit connected to the mounting box, wherein the imaging unit is arranged within the sealing protective member, and the mounting box is arranged within the mounting cavity.

2. The endoscope according to claim 1, wherein the sealing protective member is a circumferentially closed tubular member to form an imaging channel within the sealing protective member so as to allow the imaging unit to pass therein.

3. The endoscope according to claim 2, wherein a first end of the sealing protective member is arranged within the tube body and hermetically connected to an inner wall surface of a front end of the tube body; and a second end of the sealing protective member is hermetically connected to the mounting cavity.

4. The endoscope according to claim 3, wherein a closed light-transmitting part is arranged in the front end of the tube body, and the first end of the sealing protective member is abutted against the inner wall surface of the front end of the tube body.

5. The endoscope according to claim 4, wherein a connecting cap is arranged in the front end of the tube body, the connecting cap is hermetically connected to the front end of the tube body, a light-transmitting hole is arranged in the connecting cap, the light-transmitting part comprises a light-transmitting sheet, the light-transmitting sheet is provided to cover the light-transmitting hole, and an edge of the light-transmitting sheet is hermetically connected to the light-transmitting hole.

6. The endoscope according to claim 3, wherein the second end of the sealing protective member extends into the mounting cavity and is hermetically connected to an inner wall of the mounting cavity.

7. The endoscope according to claim 1, wherein the mounting box comprises a housing and a connecting seat, and a sealing cavity is within the housing, the connecting seat is arranged within the sealing cavity, the imaging unit comprises a camera and a signal transmission line, two ends of the signal transmission line are connected to the camera and the connecting seat, respectively, and the connecting seat is used for electrically connecting the signal transmission line with the first shell.

8. The endoscope according to claim 7, wherein a side wall of the housing is provided with a mounting hole for the signal transmission line to pass through, and an outer wall of the connecting seat is hermetically connected to the mounting hole.

9. The endoscope according to claim 8, wherein the connecting seat comprises a columnar part and a plate part connected to one end of the columnar part, the columnar part extends into the mounting hole, the columnar part is provided with a through-hole for the signal transmission line to pass through, the signal transmission line is hermetically connected to an inner wall of the through-hole, and a seal is provided between the columnar part and an inner wall of the mounting hole.

10. The endoscope according to claim 9, wherein the mounting box further comprises an elastic member arranged within the sealing cavity, and the elastic member is abutted between an inner wall of the housing facing towards the mounting hole and the connecting seat.

11. The endoscope according to claim 1, wherein the first shell is an integrally molded member, and a first channel, a second channel, a liquid inlet channel and a liquid outlet channel are arranged within the first shell, the first channel and the second channel are both communicated with the interior of the tube body, and at least a part of a structure of the sealing protective member is arranged within the first channel; and
the liquid inlet channel is communicated with the first channel, and the liquid outlet channel is communicated with the second channel.

12. The endoscope according to claim 11, wherein a communicating port is arranged in the front end of the first shell, the tube body is hermetically connected within the communicating port, and the first channel and the second channel are both communicated with the communicating port.

13. The endoscope according to claim 11, wherein the liquid inlet channel is communicated with a side of the first channel, and the liquid outlet channel is communicated with a side of the second channel; and/or
an inner duct is arranged within the tube body, one end of the inner duct is hermetically connected to an inner wall surface of a front end of the tube body and is communicated with an exterior of the tube body at the front end of the tube body, and the other end of the inner duct extends into the second channel and is communicated with the liquid outlet channel.

14. The endoscope according to claim 11, wherein the sealing protective member comprises a first section, a second section and a third section connected in sequence, a part of the first section is exposed to an exterior of the first shell, the second section is located within the first channel, and the third section is located within the tube body, and one end of the first channel away from the tube body is hermetically connected to an outer wall of the second section.

15. The endoscope according to claim 11, wherein one end of the first shell away from the tube body forms a fixing seat, the mounting cavity is located within the fixing seat, a disassembly port is arranged at one end of the fixing seat opposite to the sealing protective member, and the main body further comprises an end cap, the end cap being capped over the disassembly port.

16. The endoscope according to claim 15, wherein the end cap is hinged to an outer wall surface of the fixing seat, and a sealing ring is provided between the end cap and an outer peripheral surface of the fixing seat.

17. The endoscope according to claim 15, wherein the end cap is connected to the fixing seat by a resilient part, and the end cap, the fixing seat, and the resilient part are integrally formed as a monolithic structure.

18. The endoscope according to claim 15, wherein one end of the second channel away from the tube body extends into the fixing seat, the main body further comprises a waterproof sleeve, and the waterproof sleeve abuts between one end of the first channel away from the tube body and the end cap.

19. The endoscope according to claim 11, wherein the main body further comprises a second shell, the second shell comprises a protective part, a protective cavity is within the protective part, at least a part of the first shell is arranged within the protective cavity, and the liquid inlet channel and the liquid outlet channel both pass through an inner wall of the second shell and extends to an exterior of the second shell.
